(19) United States
(12) Patent Application Publication
(10) Pub. No.: US 2023/0210520 A1
(43) Pub. Date: Jul. 6, 2023

(54) DEFECT CLOSURE SYSTEM AND METHODS OF OPERATION THEREOF

(71) Applicant: St. Jude Medical, Cardiology Division, Inc., St. Paul, MN (US)

(72) Inventors: Theodore P. Dale, Corcoran, MN (US); Aaron M. Fortson, Fremont, CA (US); Timothy C. Reynolds, Sunnyvale, CA (US); Tracee Eidenschink, Wayzata, MN (US)

(21) Appl. No.: 18/148,265

(22) Filed: Dec. 29, 2022

Related U.S. Application Data
(60) Provisional application No. 63/295,234, filed on Dec. 30, 2021.

(51) Int. Cl. A61B 17/04

(52) U.S. Cl. CPC A61B 17/0469; A61B 2017/00243

(57) ABSTRACT

A method of closing a patent foramen ovale (PFO) including a septum primum and septum secundum is implemented using a suture device including an operational shaft housing an anchor assembly, a needle assembly, and a suture. The method includes advancing an end of the operational shaft to a proximal side of the PFO, positioning a distal portion of the end of the operational shaft on a distal side of the PFO, and actuating the anchor assembly to deploy the anchor assembly on the distal side of the PFO. The method also includes actuating the needle assembly on the proximal side of the PFO to deploy the needle assembly through the PFO and the anchor assembly, thereby deploying the suture through the PFO. The method further includes retracting the anchor assembly into the operational shaft, and withdrawing the distal portion of the operational shaft from the PFO, leaving the deployed suture.

### CROSS-REFERENCE TO RELATED APPLICATIONS

This application claims the benefit of priority to U.S. Provisional Patent Application No. 63/295,234, filed Dec. 30, 2021, which is hereby incorporated by reference herein in its entirety.

### BACKGROUND OF THE DISCLOSURE

### A. Field of the Disclosure

The present disclosure relates generally to a system for use in a human body, and more particularly to a defect closure system for closing at least one defect, such as an atrial septal defect (ASD).

### B. Background Art

An occluding device (or “occluder”) is a medical device used to treat tissue at a target site within the human body, such as an abnormality, a vessel, an organ, an opening, a chamber, a channel, a hole, a cavity, a lumen, or the like. For example, an occluder may be used for treating atrial septal defects (ASDs). Atrial septal defects are common congenital heart defects that allow blood to flow between the left and right atria of the heart, decreasing cardiac output.

One example type of ASD is conventionally referred to as a patent foramen ovale, or PFO. In PFOB, a hole is formed, such as during fetal development, between the left and right atria. PFOs may vary in severity from generally benign to those warranting surgical intervention, such as via implantation of an occluding device, which may be implanted in the heart to repair the PFO.

In addition to PFO interventions, other percutaneous procedures are becoming more prevalent in surgical practice as well, including those for treating a variety of atrial septal defects, including as described above, but not limited to, PFOB. Conventional devices for closing ASDs include, for example, braided devices as well as many devices that include a metallic (e.g., nitinol) frame.

However, future surgical access to left and/or right atria may be limited or otherwise made more difficult by the implantation of ASD and other occluding devices which are braided and/or have a metallic frame. Likewise, the implantation of nitinol- and metallic-frame devices may interfere with magnetic imaging techniques (e.g., MRI), as well as pose certain allergy risks (e.g., nickel allergy). In addition, implantation of some known occluders and other ASD treatment devices may also, in some cases, result in erosion of surrounding tissue and/or other disruption to the conduction system of the heart.

Accordingly, it would be desirable to close an ASD, such as a PFO, with an alternate closure device or system.

### BRIEF SUMMARY OF THE DISCLOSURE

In one aspect, a method of closing a patent foramen ovale (PFO) including a septum primum and a septum secundum is implemented using a suture device including an operational shaft housing an anchor assembly, a needle assembly, and a suture. The method includes advancing an end of the operational shaft to a proximal side of the PFO, positioning a distal portion of the end of the operational shaft on a distal side of the PFO, and actuating the anchor assembly to deploy the anchor assembly on the distal side of the PFO. The method also includes actuating the needle assembly on the proximal side of the PFO to deploy the needle assembly through the PFO and through the anchor assembly, thereby deploying the suture through the PFO. The method further includes retracting the anchor assembly into the operational shaft, and withdrawing the distal portion of the operational shaft from the PFO, leaving the deployed suture.

In another aspect, a suture device for closing a PFO includes a handle assembly comprising a first actuator and a second actuator, and an operational shaft coupled to a distal end of the handle assembly, the operational shaft housing an anchor assembly, a needle assembly, and a suture.

In a further aspect, a closure system for closing a PFO includes a suture device, a steerable catheter, and a suture trimmer. The suture device includes a handle assembly comprising a first actuator and a second actuator, and an operational shaft coupled to a distal end of the handle assembly, the operational shaft housing an anchor assembly, a needle assembly, and a suture.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a simplified diagram of a patent foramen ovale (PFO).

FIG. 2 is a perspective view of one embodiment of a closure system in accordance with the present disclosure.

FIG. 3A is a perspective view of one embodiment of a suture device for use with the closure system shown in FIG. 2. FIG. 3B is an alternative embodiment of a deployed anchor assembly of a suture device for use with the closure system shown in FIG. 2.

FIG. 4 is a side view of the suture device shown in FIG. 3A.

FIG. 5 is an additional side view of the suture device shown in FIG. 3A.

FIG. 6 is a top view of the suture device shown in FIG. 3A.

FIG. 7 is an expanded sectioned view of an anchor assembly and a needle assembly of the suture device shown in FIG. 3A.

FIG. 8 is an expanded view of the needle assembly and suture of the suture device shown in FIG. 3A.

FIG. 9 is a flow diagram of a method for closing an atrial septal defect (ASD), such as a PFO, in accordance with the present disclosure.

FIGS. 10A and 10B are a flow diagram of a first variation of the method for closing a PFO.

FIGS. 11-22 depict the steps of the first variation of the method for closing a PFO, as shown in FIGS. 10A and 10B, implemented using the suture device shown in FIGS. 3A-B, 4, 5, and 6.

FIGS. 23A and 23B are a flow diagram of a second variation of the method for closing a PFO.

FIGS. 24-27 depict the steps of the second variation of the method for closing a PFO, as shown in FIGS. 23A and 23B, implemented using the suture device shown in FIGS. 3A-B, 4, 5, and 6.

FIGS. 28A and 28B are a flow diagram of a third variation of the method for closing a PFO.

FIGS. 29-32 depict the steps of the third variation of the method for closing a PFO, as shown in FIGS. 28A and 28B, implemented using the suture device shown in FIGS. 3A-B, 4, 5, and 6.

FIG. 33 depicts a cross-sectional view of a catheter of the closure system shown in FIG. 2.

Corresponding reference characters indicate corresponding parts throughout the several views of the drawings.

### DETAILED DESCRIPTION OF THE DISCLOSURE

The present disclosure relates generally to medical devices that are used in the human body. Specifically, the present disclosure provides medical devices including suture devices for treatment of an atrial septal defect (ASD), such as a patent foramen ovale (PFO). The suture devices of the present disclosure enable closure of the ASDs without deployment of an occlusive device. As described further herein, the suture device of the present disclosure is operable in a plurality of different closure methods, such that the suture device is useable to close many different types of PFOB.

The disclosed embodiments may lead to more consistent and improved patient outcomes. It is contemplated, however, that the described features and methods of the present disclosure as described herein may be incorporated into any number of systems as would be appreciated by one of ordinary skill in the art based on the disclosure herein.

It is understood that the use of the term “target site” is not meant to be limiting, as the medical device may be configured to treat any target site, such as any vascular abnormality, a vessel, an organ, an opening, a chamber, a channel, a hole, a cavity, or the like, located anywhere in the body.

The term “vascular abnormality,” as used herein is not meant to be limiting, as the medical device may be configured to treat a variety of vascular abnormalities. Furthermore, the term “lumen” is also not meant to be limiting, as the vascular abnormality may reside in a variety of locations within the vasculature, such as a vessel, an artery, a vein, a passageway, an organ, an organ wall (e.g., an atrial septal wall), a cavity, or the like. For ease of explanation, the examples used herein refer to the closure of a patent foramen ovale, or PFO, as described herein.

The term “distal” as used herein, is a broad term and is used in its ordinary sense, including, without limitation, as in the direction of the patient, or away from a user of a device. The term “proximal” as used herein, is a broad term and is used in its ordinary sense, including, without limitation, as away from the patient, or toward the user of the device.

The present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

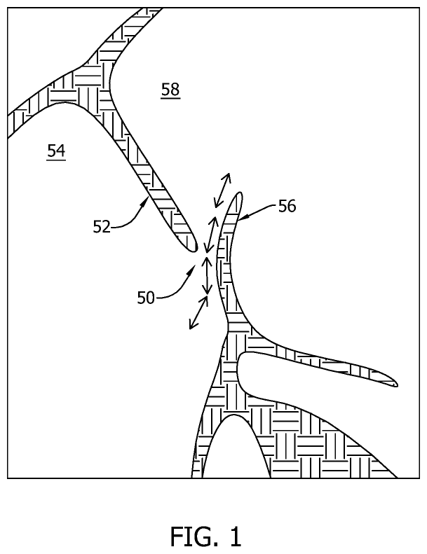

FIG. 1 illustrates an example PFO 50 in a human heart. More specifically, in a normal heart, the septum secundum of the right atrium and the septum primum of the left atrium are fused together at birth, thus forming a fossa ovalis. In contrast, as shown in FIG. 1 a PFO 50 (i.e., unformed fossa ovalis) in an open configuration is depicted. The septum secundum 52 of the right atrium 54 and the septum primum 56 of left atrium 58 overlap with one another but are not fused. During contraction/beating of the heart, the unformed fossa ovalis flaps open and shut, creating the PFO 50 that allows unwanted blood flow between the left and right atrial chambers (58 and 54).

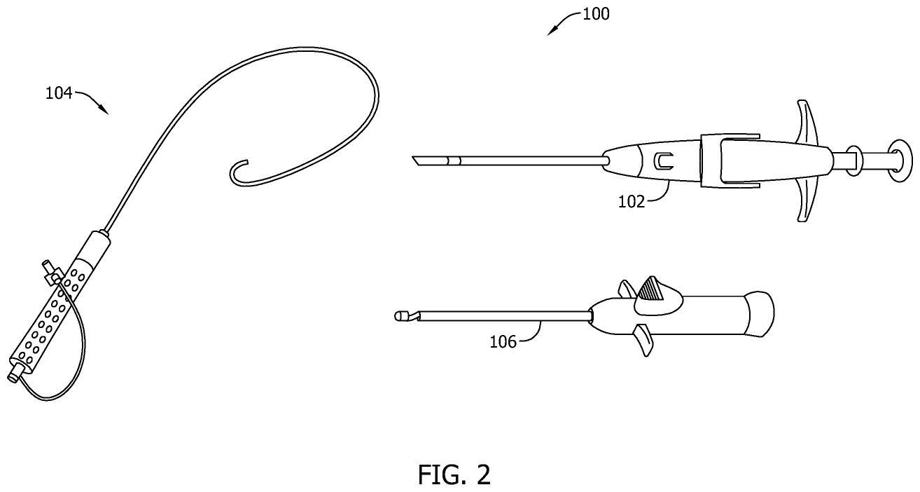

Turning to FIG. 2, a closure system 100 is depicted. The closure system includes a closure device 102, also referred to herein as a suture device 102. The suture device 102 is configured to deploy a suture at a target location, such as an ASD, and more particularly, a PFO. The closure system 100 may further include a steerable catheter 104 and a suture trimmer 106, as described further herein. Although depicted as separate from the suture device 102, the steerable catheter 104 and/or embodiments of suture trimmer 106 having a flexible shaft may, in some embodiments, be integrated into the suture device 102.

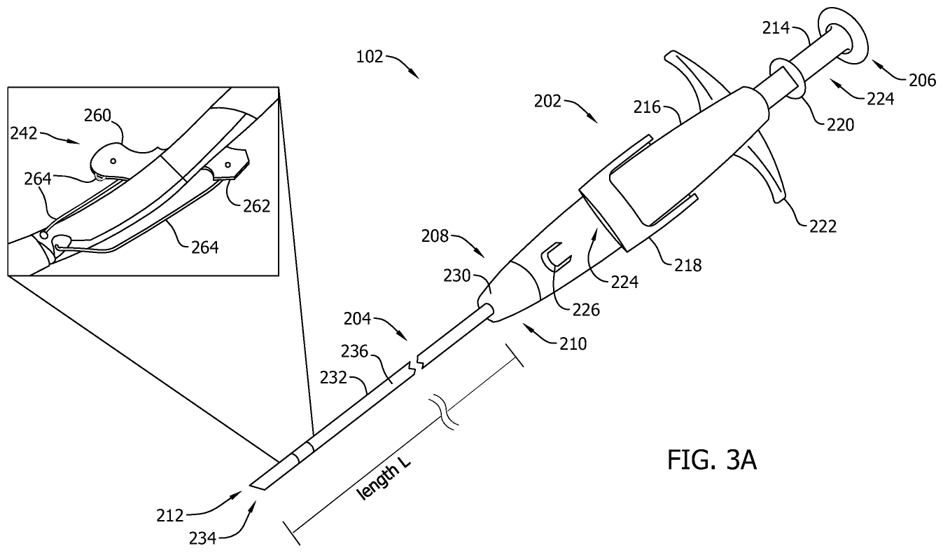

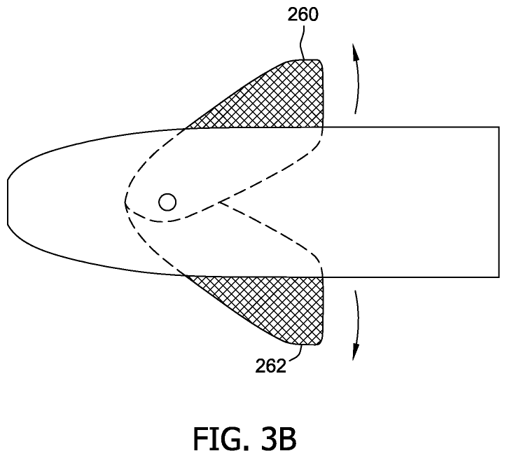

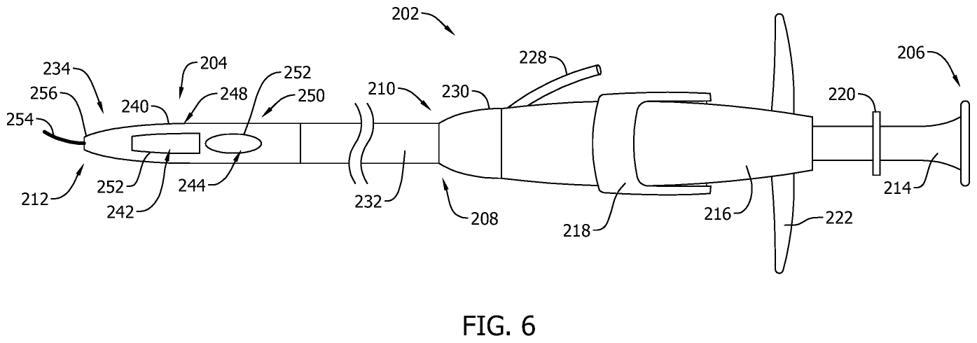

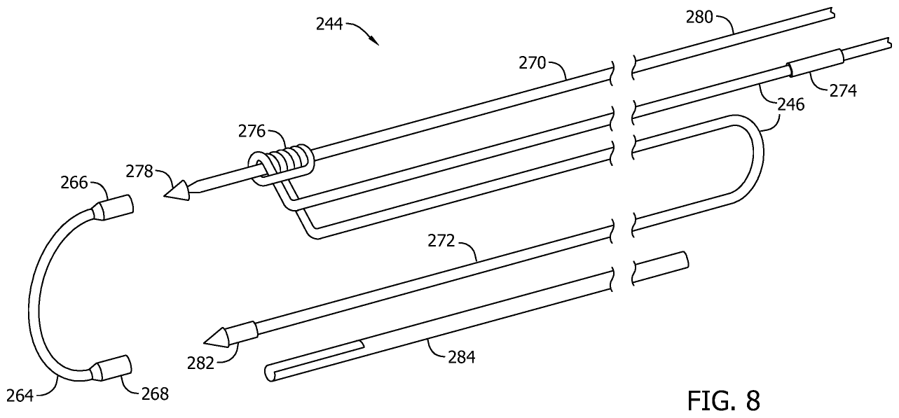

With reference to FIGS. 3A-B, 4, 5, and 6, the suture device 102 is shown in greater detail. The suture device 102 broadly includes a handle assembly 202 and an operational shaft 204. The handle assembly 202 extends from a proximal end 206 to a distal end 208, and the operational shaft 204 extends from a proximal end 210 (adjacent to the handle distal end 208) to a distal end 212. The handle assembly 202 includes a first actuator, embodied here as a plunger 214 proximal to a housing 216 of the handle assembly 202. The handle assembly 202 also includes a second actuator, embodied here as a lever 218 external to the housing 216. As described in greater detail herein, the lever 218 is manipulated to deploy an anchor assembly 242 from the operational shaft 204, and the plunger 214 is manipulated to actuate a needle assembly 244 to deploy a suture 246 (see FIG. 8) at the target location. The handle assembly 202 includes a collar 220, configured to indicate to an operator of the suture device 102 (e.g., a physician) that the plunger 214 is fully advanced into the housing 216. In some embodiments, the handle assembly 202 includes additional features, including a gripping mechanism 222 to enhance the operability of the suture device 102, instructional indicators 224 such as ordered/numbered text instructions and/or pictorial illustrations which indicate to the operator of the suture device 102 the appropriate order of steps for operating the suture device 102, and a suture trimming feature 226. The housing 216 houses internal actuation components for the actuating mechanisms described herein. In the illustrated embodiment, the housing 216 defines one or more ports 228 therein (see FIG. 6), which provide access to a lumen of the operational shaft 204 for one or more supplemental devices (e.g., a marker for locating the shaft 204 and/or the distal end 212 thereof within the vasculature of the patient).

The shaft proximal end 210 is coupled to the handle assembly distal end 208. In at least some embodiments, a strain reducer 230 is provided between the shaft 204 and the handle assembly 202, to reduce torsional strain during operation of the suture device 102. The operational shaft 204 includes a body 232 and a tip assembly 234, with which the suture 246 is deployed, as described further herein. In some embodiments, the body 232 includes a guide wire enclosed in a sheath 236 (see FIG. 3A). The sheath 236 may include one or more ports (not shown) therein, which may enable access to the shaft lumen for one or more supplemental devices (e.g., a radiopaque marker). The operational shaft 204 has a length L (see FIG. 3A) that enables navigation of the patient's vascular system to the target location, such as a PFO. In another embodiment, such as in pediatric cases, the length L of the operational shaft 204 is at least 60 cm. Accordingly, in the example embodiment, the length L of the operational shaft 204 is at least about 60 cm, at least 100 cm, or about 110 cm, or about 120 cm.

The tip assembly 234 is at the shaft distal end 212, and includes a housing 240, which houses a deployable anchor assembly 242 as well as the needle assembly 244 for deploying the suture 246 at the target site. More specifically, a distal portion 248 of the housing 240 houses the anchor assembly 242, and a proximal portion 250 (proximal to the distal portion 248) of the housing 240 houses the needle assembly 244. In different embodiments, the distal portion 248 may be spaced from the proximal portion 250 by different lengths, to accommodate PFOs of different depths.

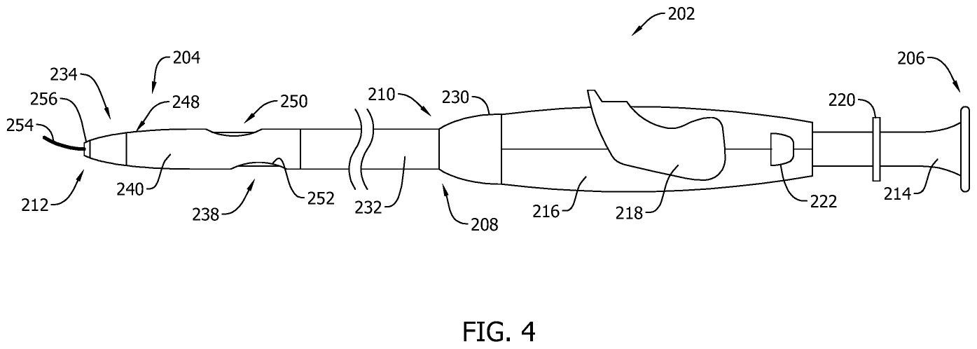

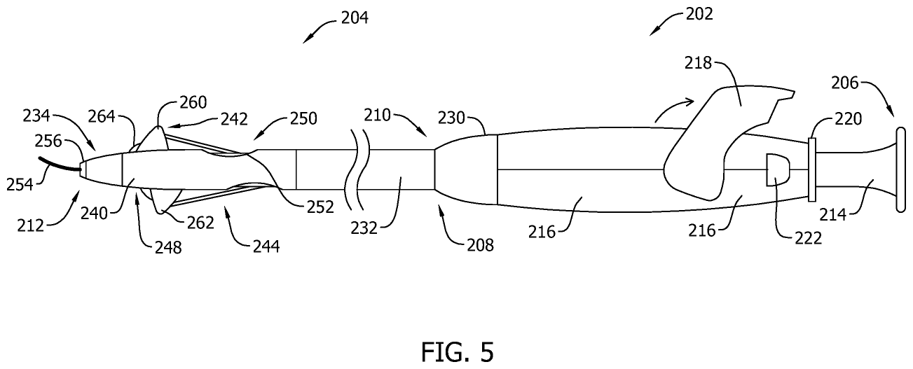

The tip assembly housing 240 includes a plurality of apertures 252 therein, which enable the anchor assembly 242 and the needle assembly 244 to be deployed from the suture device 102. FIGS. 4 and 6 depict the suture device in which the anchor assembly 242 is not deployed; FIG. 5, as well as the inset of FIG. 3A, depicts the suture device 102 with the anchor assembly 242 deployed, for actuation of the needle assembly 244 relative thereto, to deploy the suture 246 at the target location, as described in greater detail herein. In some embodiments, the tip assembly housing 240 also includes a distal aperture 256 for delivery of a guide wire 254 therethrough. In other embodiments, where a separate guidance apparatus is used to guide the tip assembly 234 and/or the shaft 204 to the target location, the shaft 204 may not include a guide wire 254 and/or the tip assembly housing 240 may not include a distal aperture 256. For example, the steerable catheter 104 may be employed to guide the tip assembly 234 to the target location.

With reference to FIGS. 3A-B, 5, and 7, the anchor assembly 242 is shown in greater detail. The anchor assembly 242 includes two locators, which are deployed from a first lever position (not shown), in which the locators are enclosed in the housing 240, to a second lever position, in which the locators are transversely deployed from the housing 240. A link 264 is coupled between a first locator 260, also referred to as an anterior locator, and a second locator 262, also referred to as a posterior locator. More particularly, a first needle capture member 266, also referred to as a needle cuff, is retained within a first passage through the first locator 260. Likewise, a second needle capture member 268 or needle cuff is retained within a second passage through the second locator 262. The link 264 is coupled between the first and second needle cuffs 266, 268. The anchor assembly 242 is operatively coupled to the lever 218 of the handle assembly 202, for instance, via one or more pull wires (not shown). In the embodiment shown in FIG. 3B, larger anchor features (i.e., first locator 260 and second locator 262) may pivot for deployment as compared to sliding for deployment as shown in the inset of FIG. 3A. The anchor assembly 242 is deployed by actuation of the lever 218, from a first lever position (shown in FIG. 4) to a second lever position (shown in FIG. 5).

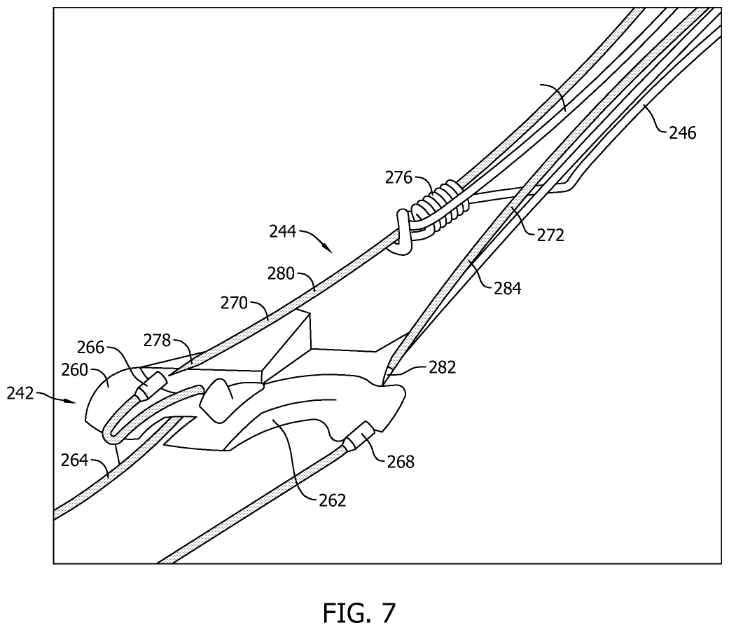

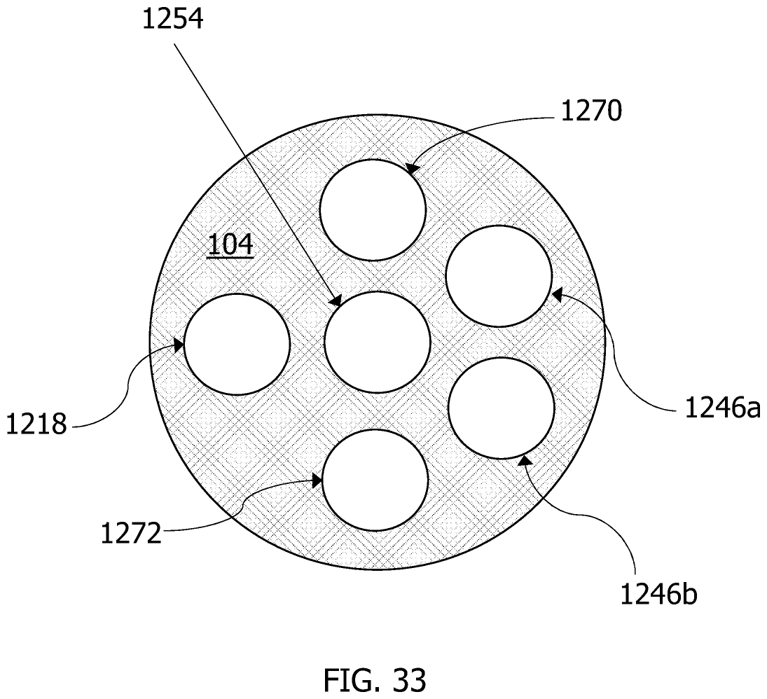

The needle assembly 244 is shown in greater detail in FIGS. 7 and 8. The needle assembly 244 includes a first or anterior needle 270, a second or posterior needle 272, a suture 246, and a suture guide 274. Depending on the embodiment, tip assembly housing 240 that houses needle assembly 244 may be sized to accommodate suture length (e.g., longer housing to accommodate longer suture lengths or shorter housing to accommodate shorter suture lengths). In some embodiments of steerable catheter 104, dedicated lumens will be housed therein to carry, for example, suture(s) 246, as well as other components that would benefit from residing within kink-resistant lumens (see also FIG. 33). In the example embodiment, the anterior needle 270 carries a suture knot 276 thereon. In other embodiments, anterior needle 270 resides within a guide assembly (not shown) which carries the suture knot 276 thereon. In embodiments that include a guide assembly, the guide assembly may include a rigid section functionable to direct anterior needle trajectory and may also house and/or assist in controlling first and second locators 260 and 262. In the example embodiment, the suture knot 276 is pre-tied and heat-set to form a preformed and permanent knot. The suture knot 276 is formed as a clinch knot; however, in alternative embodiments, other knot types may be implemented, such as a slip knot. In exemplary embodiments, suture knot 276 is formed as an improved clinch knot comprising a unique backwrap not found on a typical clinch knot. More specifically, a typical clinch knot is characterized by 5 wraps with an underpass at the anchor loop. As one of ordinary skill would recognize, an improved clinch knot provides a second underpass of the non-rail section which serves to add significant strength. The suture knot 276 is coupled to the suture guide 274 and to the suture 246, as shown in FIG. 8. The suture guide 274, suture knot 276, and suture 246 are integrally formed from a length of biocompatible suture material.

The anterior needle 270 includes a distal needle tip 278 and a needle shank 280, which extends through the suture knot 276. The posterior needle 272 also includes a distal needle tip 282 and a needle shank 284. The posterior needle tip 282 is coupled or bonded with an end of the suture 246 and is removably coupled to the posterior needle shank 284. The needle assembly 244, in particular the anterior and posterior needles (i.e., first and second needles) 270, 272, are operatively coupled to the plunger 214 of the handle assembly 202, such that the needle assembly 244 is actuated when the plunger 214 is distally depressed into the handle housing 216.

In the example embodiment, the tip assembly 234 and components thereof, including at least some portions of the anchor assembly 242 and the needle assembly 244, are constructed from a relatively flexible material, such that the tip assembly 234 can navigate the circuitous vasculature of the patient to advance the tip assembly 234 to the target location. In some embodiments, suitable materials may include nylon, PEBAX, and/or fluoropolymers. Further, some embodiments may include reinforcements such as stainless steel or nitinol braiding.

As described further herein, when the needle assembly 244 is actuated to deploy the suture 246, the anterior and posterior needles 270, 272 are driven distally. The anterior needle tip 278 engages the anterior needle cuff 266, and the posterior needle tip 282 engages the posterior needle cuff 268, where the posterior needle tip 282 de-couples from the posterior needle shank 284.

The plunger 214 is then discharged proximally from the handle housing 216 (e.g., under a biasing force of a mechanism within the handle housing 216). This movement of the plunger 214 causes corresponding proximal retraction of the anterior and posterior needles 270, 272 into the shaft 204. More specifically, the posterior needle shank 284 (de-coupled from the posterior needle tip 282) is retracted in the shaft 204, and the anterior needle 270 is retracted into the shaft 204, drawing the link 264—and, thereby, the posterior needle tip 282 and the suture 246—through the anchor assembly 242 and proximally into the shaft 204. Thereby, the suture 246 is drawn through the suture knot 276. The anchor assembly 242 is then retracted back into the operational shaft 204, and the shaft 204 is retracted, leaving the deployed suture 246 at the target location.

In some embodiments, the suture trimmer 106 is subsequently deployed to the target location to cut the suture material at a proximal end (not shown) of the suture 246. In other embodiments, a suture trimmer is integrated into the suture device 102 (e.g., into the operational shaft 204), and may be actuated to cut the suture material without requiring a separate device.

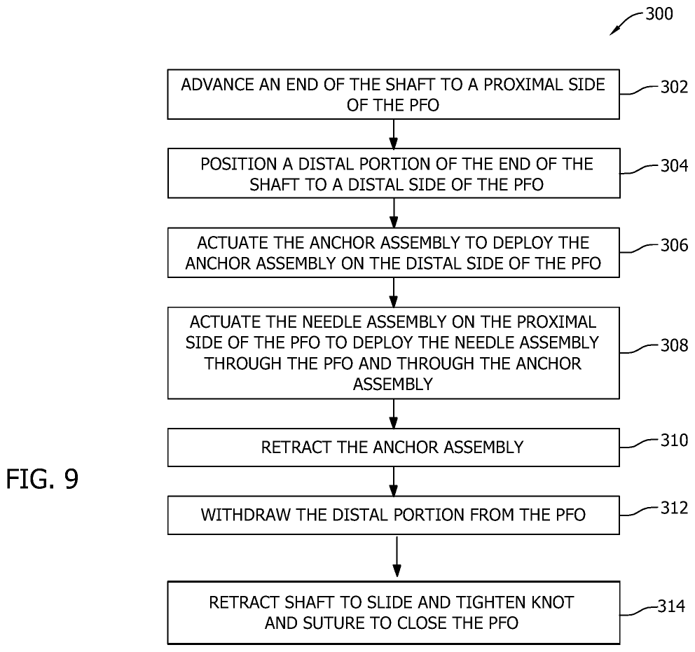

Turning to FIG. 9, a PFO closure method 300 implemented using the suture device 102 is disclosed. The method 300 includes advancing (302) an end of the operational shaft to a proximal side of the PFO, positioning (304) a distal portion of the end of the operational shaft to a distal side of the PFO, and actuating (306) the anchor assembly to deploy the anchor assembly on the distal side of the PFO. The method 300 also includes actuating (308) the needle assembly on the proximal side of the PFO to deploy the needle assembly through the PFO and through the anchor assembly, thereby deploying the suture through the PFO. The method 300 further includes retracting (310) the anchor assembly into the operational shaft, withdrawing (312) the distal portion of the operational shaft from the PFO, leaving the deployed suture, and retracting (314) the shaft to slide and tighten the knot and suture thus closing the PFO.

The method 300 may include additional, fewer, and/or alternative steps, including those disclosed elsewhere herein. For example, in some embodiments, needle assembly 244 is advanced causing anterior and posterior needles 270, 272 to advance and capture the respective first and second needle capture members (needle cuffs) 266 and 268. At an end indication of first actuator (plunger) 214 travel, posterior needle 272 is detached from posterior needle shank 284. The plunger 214 is retracted proximally until suture 246 travel ends, which places suture 246 under tension drawing link 264 and suture 246 through knot 276, thus completing the clinch (or improved clinch) knot. Anchor assembly 242 is retracted. Catheter 104 is partially withdrawn from the PFO which releases knot 276 and suture 246 from the aperture of the PFO. Additional tension is applied to suture 246 causing knot 276 to slide until knot 276 advances to the PFO. Suture 246 is tightened to lock the knot 276. Closure system 100 is retracted from steerable catheter 104 leaving the exposed suture 246. Suture trimmer 106 is loaded over suture 246 and into steerable catheter 104 and advanced to the PFO, after which suture 246 is trimmed.

The suture device 102 disclosed herein is designed for use with virtually any PFO, regardless of the PFO's particular anatomy. In particular, the function of the suture device 102 may be applied to a subject PFO in accordance with several variations, to accommodate PFOs of various sizes and shapes.

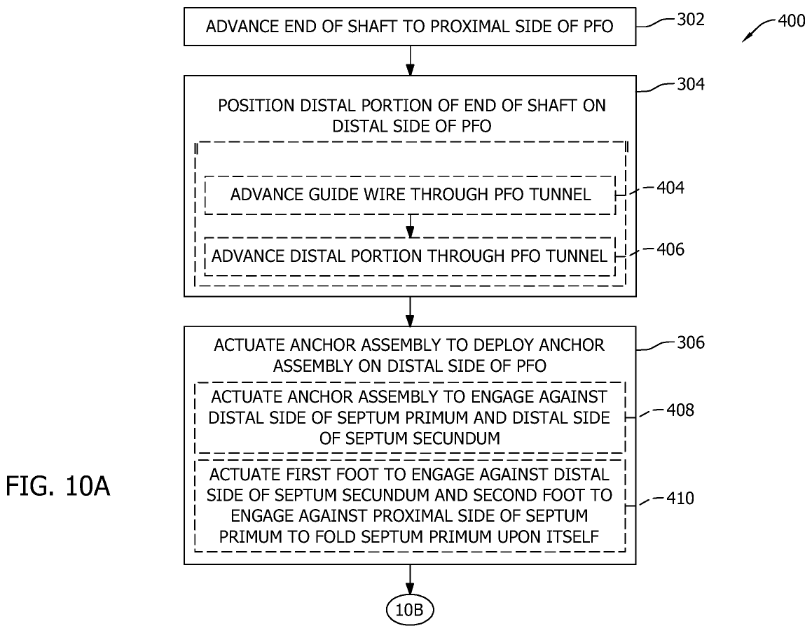

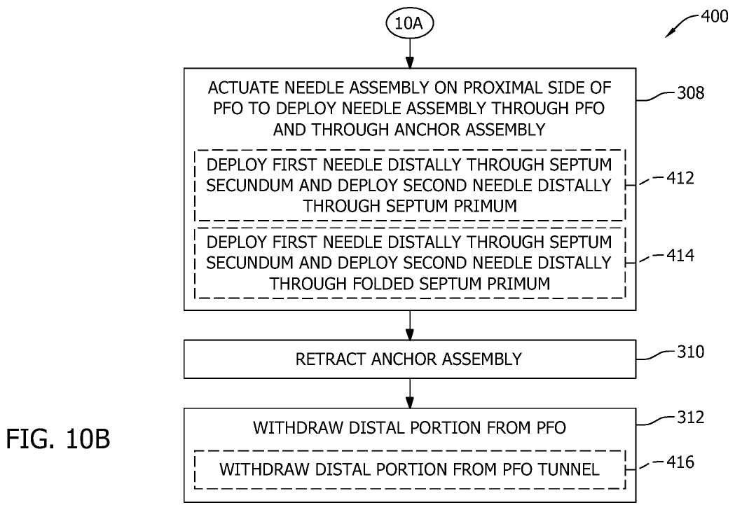

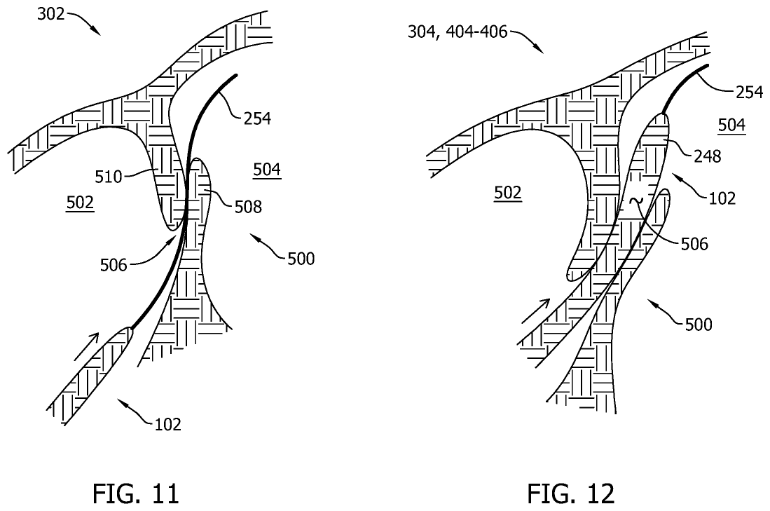

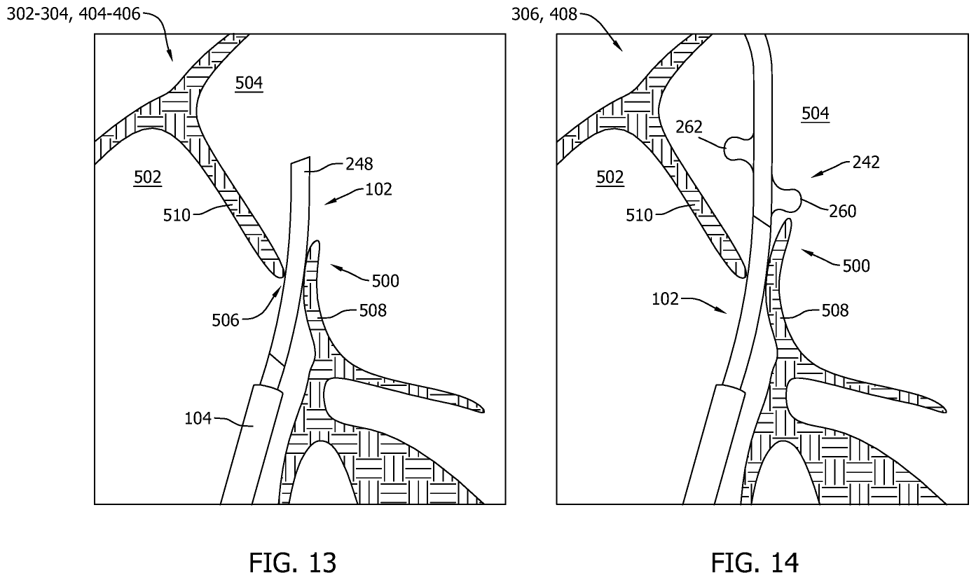

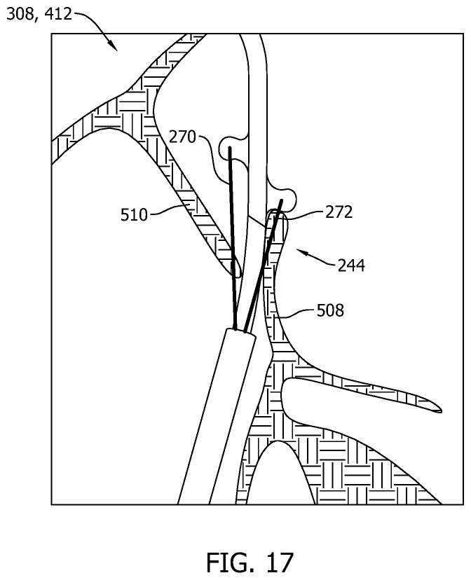

With reference to FIGS. 10A-22, a first variation 400 of the method 300 of closing a PFO using the suture device 102 is disclosed. This method variation 400 may be generally referred to as the “tunnel variation” 400. The end 212 of the operational shaft 204 is advanced (302) to the target location, specifically a PFO 500 (see FIG. 11). In some embodiments, as described herein, the operational shaft 204 may include integral steering capabilities to advance the shaft 204 to the target location. In other embodiments, as shown in FIG. 13, a separate catheter (e.g., steering catheter 104) may be used to deliver the end 212 of the shaft 204 to the target location.

In the tunnel variation 400, the distal portion 248 of the tip assembly 234 is positioned (304) on the distal side of the PFO 500 (e.g., within the left atrium 504) by advancing (404, 406) the distal portion 248 including guidewire 254 from the right atrium 502 to the left atrium 504 through a PFO tunnel 506 defined between the septum primum 508 and the septum secundum 510. In some embodiments, this positioning (304) includes advancing (404) a guide wire through the PFO tunnel 506, and advancing (406) the distal portion 248 along the guide wire through the PFO tunnel 506. The guide wire (e.g., guide wire 254) may be part of the suture device 102, and may be advanced (404) distally through the distal aperture 256 of the shaft 204. Alternatively, the guide wire may be part of a separate device (e.g., the delivery catheter 104)

As noted herein above, some embodiments may not include a guide wire 254. For example, a separate guidance apparatus may be used to guide the tip assembly 234 and/or the shaft 204 to the target location, and/or steerable catheter 104 may be employed to guide the tip assembly 234 to the target location.

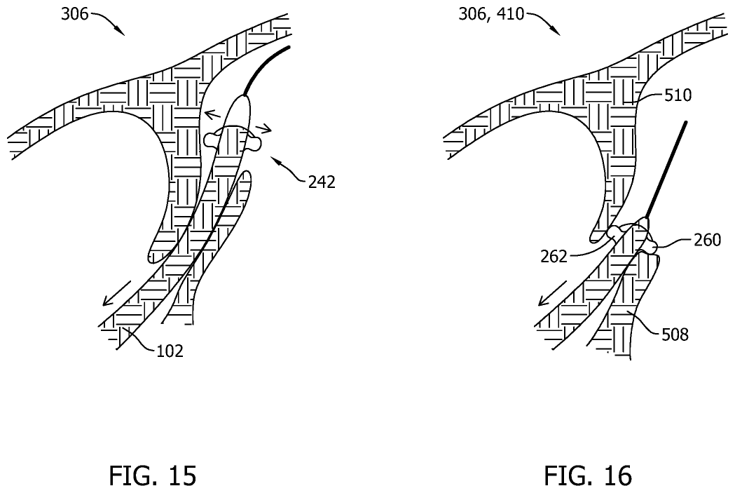

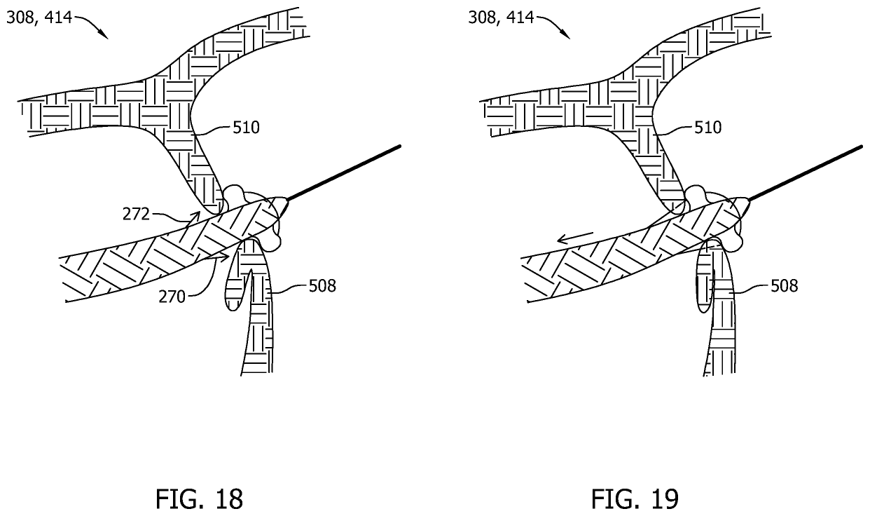

Once the distal portion 248 is positioned on the distal side of the PFO 500 (e.g., the left atrium 504), an operator of the suture device 102 actuates (306) the anchor assembly 242 (e.g., using the lever 218). Actuating (306) includes the locators 260, 262 being extended from the distal portion 248 of the tip assembly 234. In some embodiments, as shown in FIG. 14, actuating (306) also includes drawing the distal portion 248 proximally until proximal edges of the locators 260, 262 engage (408) with distal-side tissue of the PFO 500, such as a distal side of the septum primum 508 and a distal side of the septum secundum 510. In some embodiments, as shown in FIGS. 15 and 16, actuating (306) includes drawing the distal portion 248 proximally until a proximal edge of the anterior locator 260 engages (410) against a distal side of the septum primum 508 and folds the septum primum 508 upon itself (FIG. 18), and until a proximal edge of the posterior locator 262 engages (410) with a distal side of the septum secundum 510. This engagement stabilizes the distal portion 248 against the PFO tissue.

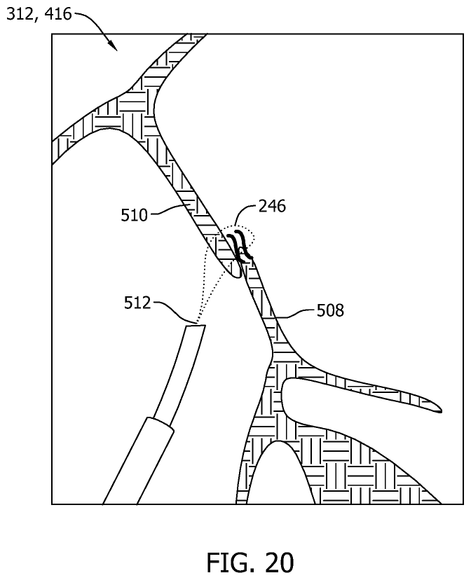

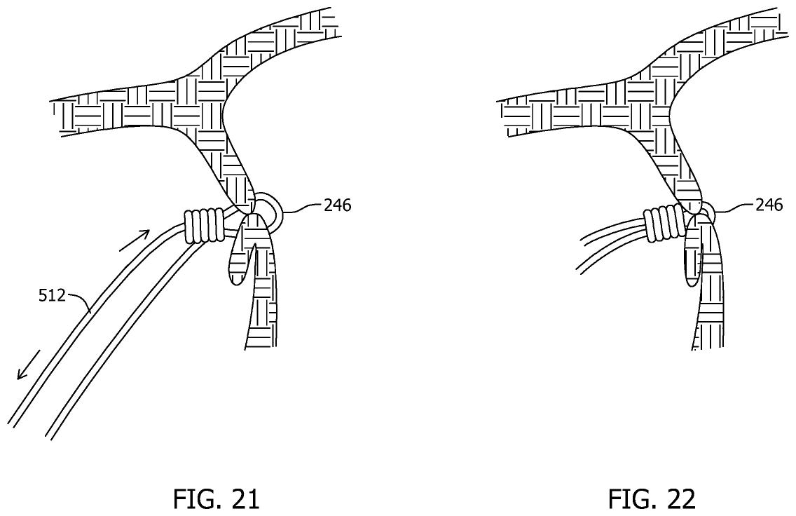

Then the needle assembly 244 is actuated (308, e.g., using the plunger 214). One needle (e.g., the anterior needle 270 or the posterior needle 272) is deployed (412) distally through the septum primum 508, and the other needle (e.g., the other of the anterior needle 270 and the posterior needle 272) is deployed (412) distally through the septum secundum 510. In embodiments where the septum primum 508 is folded upon itself, this actuating (308) includes deploying (414) one needle through the folded septum primum 508. The needle assembly 244 operates as described above to draw the suture 246 through the PFO tissue, specifically, distally through one of the septum primum 508 and the septum secundum 510 and proximally through the other of the septum primum 508 and the septum secundum 510. The distal portion 248 is withdrawn back through the PFO tunnel 506, and the suture 246 is tightened to engage the septum primum 508 tightly against the septum secundum 510, to secure the PFO 500 in a closed configuration. A proximal end 512 (see FIGS. 20 and 21) of the suture material is cut, leaving the suture 246 deployed in the PFO 500 (see FIG. 22).

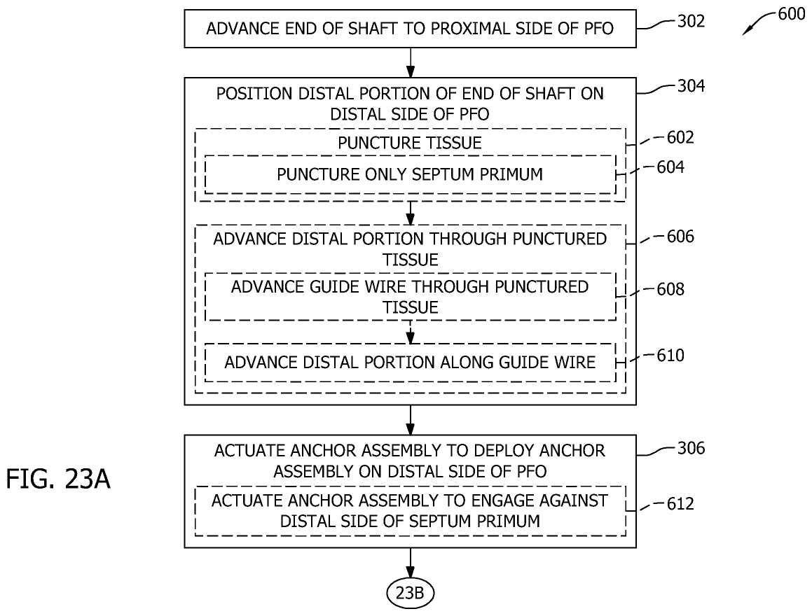

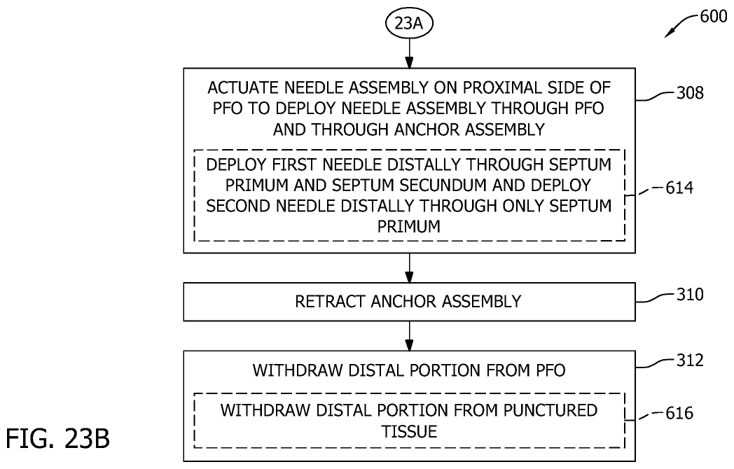

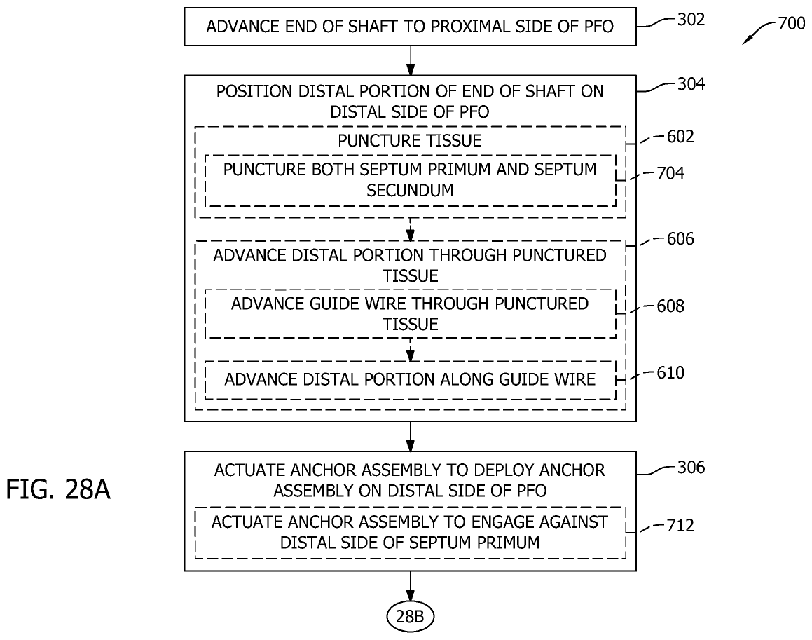

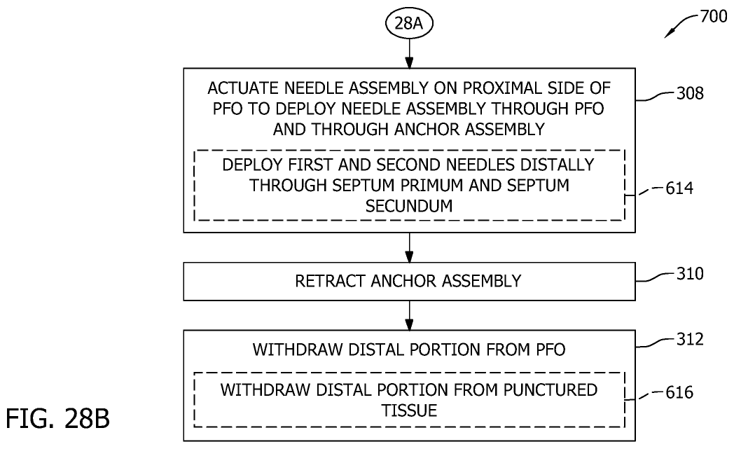

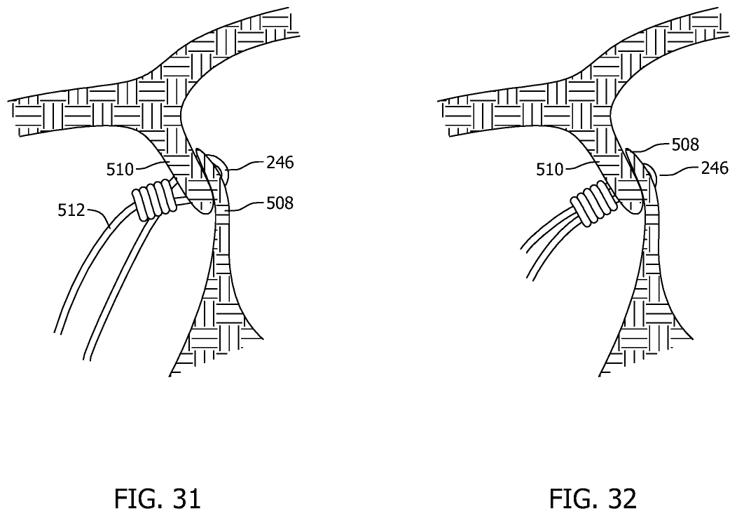

With reference to FIGS. 23A-27, a second variation 600 of the method 300 of closing a PFO (e.g., the PFO 500) using the suture device 102 is disclosed, and in FIGS. 28A-32, a third variation 700 of the method 300 is disclosed. Both the second and third variations 600, 700 may be generally referred to as “puncture variations.” More particularly, the second variation 600 may be referred to a “single puncture variation,” and the third variation 700 may be referred to as “double puncture variation.”

Here again, some “single puncture variation” and “double puncture variation” embodiments may not include a guide wire 254. For example, a separate guidance apparatus may be used to guide the tip assembly 234 and/or the shaft 204 to the target location, and/or steerable catheter 104 may be employed to guide the tip assembly 234 to the target location.

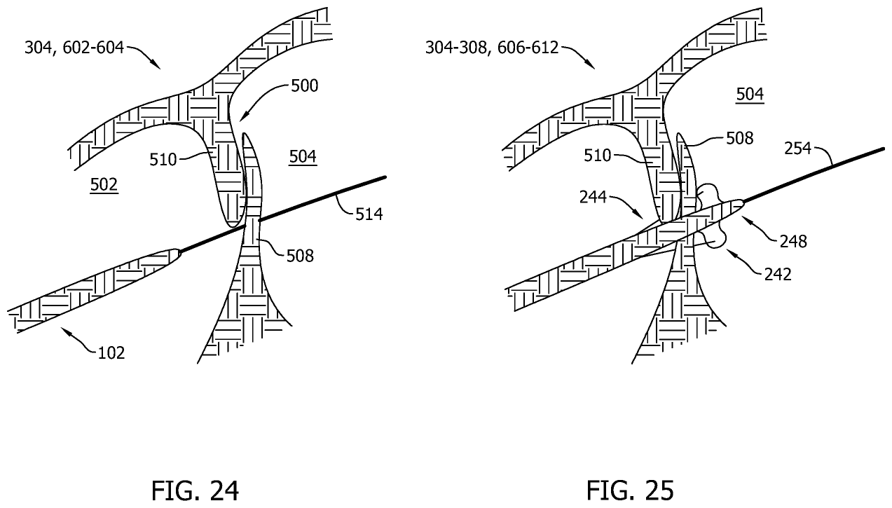

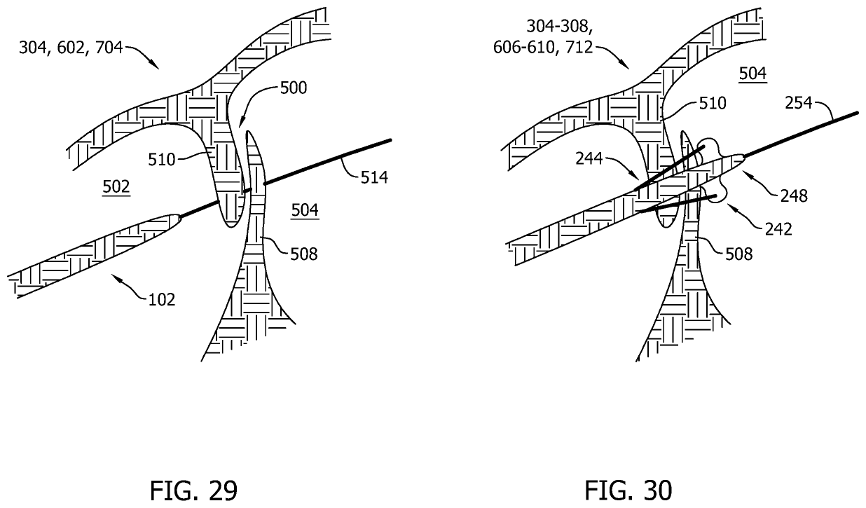

In the puncture variations 600, 700, the end 212 of the operational shaft 204 is advanced (302) to the target location, specifically to the PFO 500. Positioning (304) the distal portion 248 of the end 212 of the operational shaft 204 on the distal side (e.g., the left atrium 504) of the PFO 500 includes puncturing (602) a portion of the tissue of the PFO 500 and advancing (606) the distal portion 248 through the punctured tissue. The puncture may be made using a supplemental component of the suture device 102 (e.g., a puncture needle 514, see FIGS. 24 and 29), or using a separate device (e.g., delivered to the target site using the delivery catheter 104).

More specifically, in the single puncture variation 600, the puncture is made (604) through only the septum primum 508. A guide wire 254 may be advanced (608) through the punctured tissue, and the distal portion 248 may be advanced (610) to the distal side of the PFO 500 (e.g., the left atrium 504) along the guide wire 254. The guide wire 254 may be part of the suture device 102, and advanced distally through the distal aperture 256 of the shaft 204. Alternatively, the guide wire 254 may be part of a separate device (e.g., the delivery catheter 104).

Once the distal portion 248 is positioned (304) on the distal side of the PFO 500, an operator of the suture device 102 actuates the anchor assembly 242 (e.g., using the lever 218). The locators 260, 262 are extended from the distal portion 248 of the tip assembly 234. The distal portion 248 is drawn proximally until proximal edges of the locators 260, 262 engage with distal-side tissue of the PFO 500; in particular, the locators 260, 262 engage (612) against a distal side of the septum primum 508. This engagement stabilizes the distal portion 248 against the PFO tissue.

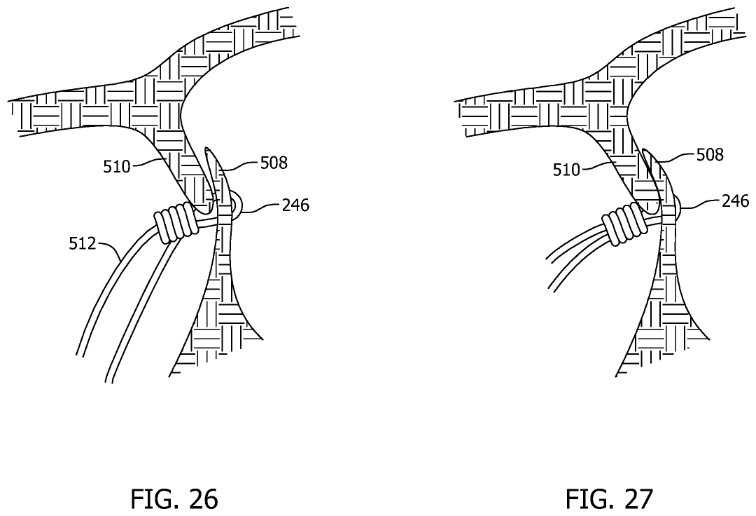

Then the needle assembly 244 is actuated (308; e.g., using the plunger 214). One needle (e.g., the anterior needle 270 or the posterior needle 270) is deployed (614) distally through a first location in the septum primum 508, and the other needle (e.g., the other of the anterior needle 270 and the posterior needle 272) is deployed (614) distally through both the septum secundum 510 and a second location in the septum primum 508. The needle assembly 244 operates as described above to draw the suture 246 through the PFO tissue, specifically distally through the septum primum 508 and proximally through both the septum primum 508 and the septum secundum 510. In an alternative embodiment, the suture 246 is drawn through the PFO 500 in the opposite way, that is, distally through both the septum primum 508 and the septum secundum 510 and proximally back through the septum primum 508. The anchor assembly 242 is retracted (310) back into the shaft 204, and the distal portion 248 is withdrawn (312, 616) back through the punctured PFO tissue (e.g., the septum primum 508). The suture 246 is tightened to engage the septum primum 508 tightly against the septum secundum 510, to secure the PFO 500 in a closed configuration. A proximal end 512 of the suture material is cut, leaving the suture 246 deployed in the PFO 500 (see FIG. 27).

In the double puncture variation 700, the puncture is made (704) through both of the septum primum 508 and the septum secundum 510. A guide wire 254 may be advanced (608) through the punctured tissue, and the distal portion 248 may be advanced (610) to the distal side of the PFO 500 (e.g., the left atrium 504) along the guide wire 254. The guide wire 254 may be part of the suture device 102, and advanced distally through the distal aperture 256 of the shaft 204. Alternatively, the guide wire 254 may be part of a separate device (e.g., the delivery catheter 104).

Once the distal portion 248 is positioned (304) on the distal side of the PFO 500, an operator of the suture device 102 actuates the anchor assembly 242 (e.g., using the lever 218). The locators 260, 262 are extended from the distal portion 248 of the tip assembly 234. The distal portion 248 is drawn proximally until proximal edges of the locators 260, 262 engage with distal-side tissue of the PFO 500; in particular, the locators 260, 262 engage (712) against as a distal side of the septum primum 508. This engagement stabilizes the distal portion 248 against the PFO tissue.

Then the needle assembly 244 is actuated (308; e.g., using the plunger 214). Both needles 270, 272 are deployed (714) distally through both the septum secundum 510 and the septum primum 508. The needle assembly 244 operates as described above to draw the suture 246 through the PFO tissue, specifically distally through a first location in both the septum primum 508 and the septum secundum 510 and proximally through a second location in both the septum primum 508 and the septum secundum 510. The anchor assembly 242 is retracted (310) back into the shaft 204, and the distal portion 248 is withdrawn (312, 616) back through the punctured PFO tissue (e.g., the septum primum 508 and the septum secundum 510). The suture 246 is tightened to engage the septum primum 508 tightly against the septum secundum 510, to secure the PFO 500 in a closed configuration. A proximal end 512 of the suture material is cut, leaving the suture 246 deployed in the PFO 500 (see FIG. 32).

In exemplary embodiments, the length of steerable catheter 104 enables suture storage in lumens within the catheter 104 (e.g., within handle assembly 202 and/or operational shaft 204). FIG. 33 shows a cross-sectional view of steerable catheter 104 including several lumens such as, though not limited to, a first needle lumen 1270, suture lumens 1246a and 1246b, a second needle lumen 1272, a second actuator lumen 1218, and a guidewire lumen 1254. In some embodiments, the portion of steerable catheter 104 housing one or more lumens may be formed by separate extrusions supported by an outer sheath with spring coils or braids to prevent kinking. Depending on the embodiment, sub-portions of steerable catheter 104 housing one or more lumens may be rigid and interconnected with the other, flexible portions of catheter 104.

The suture device disclosed herein presents advantages over conventional PFO closure medical devices, such as occluders, as the suture device reduces or eliminates the presence of metal in the body, deploying only a biocompatible suture. The suture device includes particular features, including the long operational shaft and the optional incorporated steering/navigating functionality and trimming functionality, that enable use of the suture device in percutaneous procedures, specifically for the closure of ASDs including PFOB. Further features include a flexible, kink-resistant multi-lumen based catheter shaft, for example with braided or coiled support, larger anchor (i.e., locator) features that pivot rather than slide for deployment, increased locator area for proper needle capture, fully over-the-wire (OTW) rather than guidewire exchange as appreciated by those in the art, and exemplary embodiments having a suture length exceeding 110 inches (260 cm) in a 130 cm closure system.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A method of closing a patent foramen ovale (PFO) including a septum primum and a septum secundum, the method implemented using a suture device including:
an operational shaft housing an anchor assembly, a needle assembly, and a suture, the method comprising:
advancing an end of the operational shaft to a proximal side of the PFO;
positioning a distal portion of the end of the operational shaft on a distal side of the PFO;
actuating the anchor assembly to deploy the anchor assembly on the distal side of the PFO;
actuating the needle assembly on the proximal side of the PFO to deploy the needle assembly through the PFO and through the anchor assembly, thereby deploying the suture through the PFO;
retracting the anchor assembly into the operational shaft; and
withdrawing the distal portion of the operational shaft from the PFO, leaving the deployed suture.

2. The method of claim 1, further comprising tightening the suture to engage the septum primum tightly against the septum secundum, to secure the PFO in a closed configuration.

3. The method of claim 1, further comprising cutting a proximal end of the suture.

4. The method of claim 1, wherein positioning the distal portion of the end of the operational shaft on the distal side of the PFO comprises advancing the distal portion through a PFO tunnel defined between the septum primum and the septum secundum, and
wherein actuating the anchor assembly comprises actuating the anchor assembly to engage against a distal side of the septum primum and a distal side of the septum secundum.

5. The method of claim 4, wherein advancing the distal portion through the PFO tunnel comprises:
advancing a guide wire through the PFO tunnel; and
advancing the distal portion along the guide wire through the PFO tunnel.

6. The method of claim 5, wherein advancing the guide wire comprises advancing the guide wire distally from the end of the operational shaft and through the PFO tunnel.

7. The method of claim 5, wherein advancing the guide wire comprises advancing a guide wire separate from the suture device through the PFO tunnel.

8. The method of claim 4, wherein actuating the needle assembly comprises:
deploying a first needle distally through the septum secundum; and
deploying a second needle distally through the septum primum.

9. The method of claim 4, wherein actuating the anchor assembly comprises:
actuating a first locator of the anchor assembly to engage against a distal side of the septum secundum; and
actuating a second locator of the anchor assembly to engage against a proximal side of the septum primum and fold the septum primum distally upon itself, and
wherein actuating the needle assembly comprises:
deploying a first needle distally through the septum secundum; and
deploying a second needle distally through the folded septum primum.

10. The method of claim 4, wherein withdrawing the distal portion from the PFO comprises withdrawing the distal portion proximally through the PFO tunnel.

11. The method of claim 1,
wherein positioning the distal portion of the end of the operational shaft on the distal side of the PFO comprises:
puncturing a portion of tissue of the PFO including at least one of the septum primum or the septum secundum; and
advancing the distal portion through the punctured PFO tissue, and
wherein advancing the distal portion through the punctured PFO tissue comprises:
advancing a guide wire through the punctured PFO tissue; and
advancing the distal portion along the guide wire through the punctured PFO tissue.

12. The method of claim 11, wherein advancing the guide wire comprises advancing the guide wire distally from the end of the operational shaft and through the punctured PFO tissue.

13. The method of claim 11, wherein puncturing a portion of the PFO tissue comprises puncturing only the septum primum.

14. The method of claim 13, wherein actuating the anchor assembly comprises actuating the anchor assembly to engage against a distal side of the septum primum, and
wherein actuating the needle assembly comprises:
deploying a first needle distally through the septum primum and the septum secundum; and
deploying a second needle distally through only the septum primum.

15. The method of claim 11, wherein puncturing a portion of the PFO tissue comprises puncturing both of the septum primum and the septum secundum.

16. The method of claim 15, wherein actuating the anchor assembly comprises actuating the anchor assembly to engage against a distal side of the septum primum, and
wherein actuating the needle assembly comprises:
deploying a first needle distally through both the septum primum and the septum secundum; and
deploying a second needle distally through both the septum primum and the septum secundum.

17. A suture device for closing a patent foramen ovale (PFO), the device comprising:
a handle assembly comprising a first actuator and a second actuator; and
an operational shaft coupled to a distal end of the handle assembly, the operational shaft housing an anchor assembly, a needle assembly, and a suture, wherein the first actuator is manipulable to actuate the needle assembly and wherein the second actuator is manipulable to actuate the anchor assembly.

18. The suture device of claim 17, wherein the operational shaft has a length of at least about 60 cm, at least 100 cm, or about 110 cm, or about 120 cm.

19. The suture device of claim 17, wherein the needle assembly is distal to the anchor assembly.

20. The suture device of claim 17, wherein the first actuator is a plunger such that distal movement of the plunger deploys the needle assembly and proximal movement of the plunger retracts the needle assembly, and wherein the second actuator is a lever such that a first lever position corresponds to the enclosed position of the anchor assembly and a second lever position corresponds to the transversely deployed position of the anchor assembly.

21. The suture device of claim 17, wherein the anchor assembly comprises a first locator and a second locator, and wherein the first and second locators are moveable between an enclosed position and a transversely deployed position.

22. The suture device of claim 21, further comprising a link coupled between the first locator and the second locator.

23. The suture device of claim 17, wherein the needle assembly comprises a first needle and a second needle, and wherein the suture comprises a suture knot.
